(19) United States
(12) Patent Application Publication
(10) Pub. No.: US 2010/0228225 A1
(43) Pub. Date: Sep. 9, 2010

(54) METHOD AND SYSTEM FOR DELIVERY OF NEUROTOXINS

(76) Inventor: David Cipolla, San Ramon, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/161,724

(22) PCT Filed: Feb. 16, 2007
(86) PCT No.: PCT/US07/04201

§ 371 Date: Aug. 28, 2008

Related U.S. Application Data
(60) Provisional application No. 60/774,458, filed on Feb. 17, 2006.

(51) Int. Cl. A61M 5/00

(52) U.S. Cl. 604/506; 604/173

(57) ABSTRACT

A method and system for delivering a neurotoxin into the body of a subject is disclosed. The method includes sequentially applying one or more arrays of micro needles on a portion of the skin of the subject that requires treatment. The micro needles are pre-coated with the neurotoxin on an array comprised of 10 to 1,000 micro needles over an area of 1 mm² to 4 cm² with each micro needle having a length of from 1 micron to about 1,000 microns. The sequential application of the one or more arrays of micro needles on the portion of the skin of the subject results in delivery of the neurotoxin into the subject.

### CROSS REFERENCE

This application is a 371 National Phase of International Application Serial No. PCT/US2007/004201, filed Feb. 16, 2007, which application claims the benefit of priority to U.S. Patent Provisional Patent Application Ser. No. 60/774,458, filed Feb. 17, 2006 which are incorporated herein by reference in their entirety noting that the current application controls to the extent there is any contradiction with any earlier applications and to which applications we claim priority under 35 USC §120 and §119.

### FIELD OF THE INVENTION

The present invention relates generally to devices and methods for the delivery of neurotoxins into the body of a subject and more specifically to the delivery of the neurotoxins, using an array of micro needles or micro projectiles.

### BACKGROUND OF THE INVENTION

A neurotoxin is a drug which may be used for the treatment of disorders, such as axillary hyperhidrosis, palmar hyperhidrosis, warts, corns, calluses, neuromas, ulcers, hammertoes, bunions, and post herpetic neuralgia. To treat these disorders, a neurotoxin is delivered into the body of a subject who is affected by these disorders. Existing methods for delivering neurotoxins include delivery through an injection and delivery through oral administration. Examples of injections include standard needle and syringe, intradermal injections, hypodermic injections. Neurotoxins may also be delivered using transdermal patches.

Medical expertise is required to deliver a neurotoxin through injection. Further, the delivery through injection may cause local pain, and exposure of the subject to blood-borne diseases. In addition, the delivery of an adequate amount of the neurotoxin may require many injections, and extensive time for preparation and administration of the injections. Moreover, injections may not be able to deliver the neurotoxin at the desired rates. In the case of oral administration, bioavailability of the neurotoxin to the body depends on the pH of the subject's stomach. The bioavailability may be reduced when the pH is abnormally acidic.

There is a need for a method for delivering a neurotoxin that does not require medical expertise. The method should ensure less local pain than that of the existing methods of delivering a neurotoxin. Further, the method should be able to deliver a neurotoxin at the desired rates. Moreover, the method should retain neurotoxin's bioavailability for a therapeutic effect. Moreover, the method should reduce the treatment time, number of dosages of a neurotoxin and the possibility of blood contamination.

### SUMMARY OF THE INVENTION

A method of delivering a neurotoxin to a patient subject is disclosed. The method comprises using an array of micro needles which are pre-coated with a neurotoxin. The array is brought into contact with the surface to which the neurotoxin is to be administered. For example, the points of the microarray needles are moved through the skin of the subject. A plurality of arrays of micro needles having neurotoxins on the needles can be sequentially brought into contact with the surface to which the neurotoxin is to be administered. The neurotoxin may include any known or later developed neurotoxin such as botulinum toxin and the needles can be applied to the area being treated for a period of from about 1 second to about 30 minutes or 5 seconds to 10 minutes or 10 seconds to 5 minutes or 20 seconds to 1 minute. The micro needles in the array may have a length in a range of from about 1 micron to about 1 thousand microns or 50 microns to about 400 microns. Depending on the amount of neurotoxin to be administered the micro needles may be coated over their entire surface (100%) or over any portion of their surface, e.g. 1% of the micro needle surface. The needles may be solid, i.e. not hollow needles.

The device of the invention comprises the array of micro needles which array may be of any size, configuration or micro needle density. However, the array preferably comprises micro needles having a length of between 1 micron and 1,000 microns or 50 microns to 400 microns over an area of between about 1 mm² to about 200 cm² comprising 2 to 2,000 needles per square centimeter or 20 to 200 per square centimeter. The micro needle arrays may be prepared in kits which kits can contain one array to any number of arrays such as 1 to 25 or 1 to 10. When a plurality of arrays are included the arrays are applied to the target area sequentially over a given period of time with each array being administered for about 1 second to about 30 minutes or about 5 seconds to about 10 minutes.

An aspect of the invention is to provide a method and device which does not require substantial medical expertise for administration.

Another aspect of the invention is to provide a method which does not cause substantial pain to the patient during administration by using narrow gauge and short length micro needles.

Another aspect of the invention is to provide a method whereby neurotoxins may be delivered in a desired amount and rate closely matched based on the amount of neurotoxin on the needles and the period of time during which the needles are brought into contact with the desired target area.

Yet another aspect of the invention is to provide for the delivery of neurotoxin in an amount so as to provide sufficient bioavailability for the desired therapeutic effect.

Another aspect of the invention is to provide a method which reduces the treatment time, the number of doses of neurotoxin and reduces the possibility of blood contamination.

Another aspect of the invention is a method of administering a neurotoxin to a target area of a patient, comprising the steps of:

(a) applying an array of micro needles to a target area of a patient wherein the micro needles are coated with a therapeutically effective amount of a formulation comprised of a neurotoxin; and

(b) allowing the micro needles to remain in the target area for a period of time such that neurotoxin is delivered to the target area.

Yet another aspect of the invention is a method further comprising:

diagnosing the subject as having a disorder selected from a group consisting of axillary hyperhidrosis, palmar hyperhidrosis, warts, corns, calluses, neuromas, ulcers, hammertoes, bunions and post herpetic neuralgia; and

(c) repeating steps (a) and (b) a plurality of times using a new array of micro needles in each step (a).

These and other aspects of the invention will become apparent to those skilled in the art upon review of this disclosure including the figures as described below.

### BRIEF DESCRIPTION OF THE FIGURES

Various embodiments of the invention will hereinafter be described in conjunction with the appended drawings that have been provided to illustrate, and not limit the invention, wherein like designations denote like elements, and in which:

FIG. 1 illustrates an array of micro needles for delivering a neurotoxin, in accordance with an embodiment of the invention; and

FIG. 2 is a flowchart of a method for delivering a neurotoxin into a subject, in accordance with an embodiment of the invention.

### DETAILED DESCRIPTION OF THE INVENTION

Before the present method and devices are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a micro needle” includes a plurality of such micro needle and reference to “the neurotoxin” includes reference to one or more neurotoxins and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Neurotoxin

The neurotoxin may be any neurotoxin and can be one commercially available. The neurotoxin may be a polypeptide, comprising:

(a) a first amino acid sequence region comprising a wild type neuronal binding moiety, substantially completely derived from a neurotoxin selected from a group consisting botulinum toxin types A, B, C₁, D, E, F, G and mixtures thereof;

(b) a second amino acid sequence region effective to translocate the polypeptide or a part thereof across an endosome membrane, and;

(c) a third amino acid sequence region having therapeutic activity when released into a cytoplasm of a target cell.

The first amino acid sequence region of the polypeptide can comprise a carboxyl terminal of a heavy chain derived from the neurotoxin and the neurotoxin can be a botulinum toxin, such as botulinum toxin type A.

The second amino acid sequence region of the polypeptide can have an amine terminal of a heavy chain derived from a neurotoxin selected from a group consisting of botulinum toxin types A, B, C₁, D, E, F, G and mixtures thereof. Notably, the second amino acid sequence region of the polypeptide can include an amine terminal of a toxin heavy chain derived from botulinum toxin type A.

Finally, the third amino acid sequence region of the polypeptide can comprise a toxin light chain derived from a neurotoxin selected from a group consisting of Clostridium beratti toxin; butyricum toxin; tetani toxin; botulinum toxin types A, B, C₁, D, E, F, G and mixtures thereof. The third amino acid sequence region of the polypeptide can include a toxin light chain derived from botulinum toxin type A.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of .gtoreq.3×10⁷ U/mg, an A₂₆₀/A₂₇₈ of less than 0.60 and a distinct pattern of banding on gel electrophoresis. The known Shantz process can be used to obtain crystalline botulinum toxin type A, as set forth in Shantz, E. J., et al, Properties and Use of Botulinum Toxin and Other Microbial Neurotoxins in Medicine, Microbiol Rev. 56; 80-99:1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

Botulinum toxins and/or botulinum toxin complexes can be obtained from List Biological Laboratories, Inc., Campbell, Calif.; the Centre for Applied Microbiology and Research, Porton Down, U. K.; Wako (Osaka, Japan), Metabiologics (Madison, Wis.) as well as from Sigma Chemicals of St. Louis, Mo.

Pure botulinum toxin is so labile that it is generally not used to prepare a pharmaceutical composition. Furthermore, the botulinum toxin complexes, such as the toxin type A complex are also extremely susceptible to denaturation due to surface denaturation, heat, and alkaline conditions. Inactivated toxin forms toxoid proteins which may be immunogenic. The resulting antibodies can render a patient refractory to toxin injection.

### Micro Needle Arrays

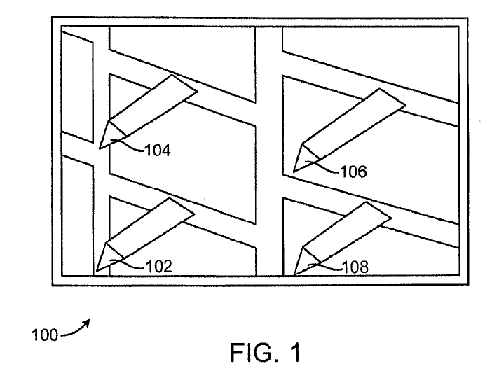

FIG. 1 illustrates an array of micro needles 100 for delivering a neurotoxin, in accordance with an embodiment of the invention. Array of micro needles 100 is used to deliver the neurotoxin a target site of a subject. The neurotoxin is delivered to the target site of the subject to treat disorders, such as axillary hyperhidrosis, palmar hyperhidrosis, warts, corns, calluses, neuromas, ulcers, hammertoes, bunions, and post herpetic neuralgia.

The array of micro needles 100 includes one or more (a plurality of) micro needles pre-coated with the neurotoxin. In an embodiment of the invention, the number of micro needles is in the range of 10 to 1000 per 10 mm² to 2 cm². In an embodiment of the invention, array of micro needles 100 includes a micro needle 102, a micro needle 104, a micro needle 106 and a micro needle 108. Micro needles 102, 104, 106, and 108 may be arranged in various configurations, such as a matrix (columns and rows), a circle, a square, a rectangle, an oval, or an ellipsoid to form an array. However, the invention should not be construed to be limited to the use of only the above-mentioned configurations of micro needles in array of micro needles 100; other configurations may be used without deviating from the scope of the invention.

Further, an array of micro needles 100 may have various sizes. In an embodiment of the invention, the size of array of micro needles 100 is in the range of 1 mm² to 200 cm² or 1 mm² to 25 cm². The size of array of micro needles 100 may be related to the distance between any two micro needles of the array. The micro needles, for example, micro needles 102 and 104 may be placed at a pre-determined distance from each other and each needle may be the same distance away from its next closest needle. The pre-determined distance between micro needles 102 and 104 may be selected, based on the amount of the neurotoxin required to be administered into the body of the subject. Micro needles 102 and 104 should be substantially close to each other to ensure the affected surface of the subject's skin is dosed with the neurotoxin. Micro needles 102 and 104 should not be so far apart that the neurotoxin is unable to spread over the surface of the skin that requires treatment. The micro needles should also not be closer than necessary, as the array of micro needles 100 should be designed to minimize the number of micro needles required to treat the affected surface of the skin.

Those skilled in the art will understand that a range of different types of arrays of micro needles may be used in connection with the present invention. For example, applicants refer to U.S. Pat. No. 6,256,533 issued Jul. 3, 2001 which discloses different types of microarrays which could be used in connection with the present invention. Applicants also refer to U.S. Pat. No. 6,790,372 issued Sep. 14, 2004. The micro needles of the array may be of the type which are hollow needles through which drug can be injected. However, the needles may be solid and the drug coated on the surface of the needles. As shown within the patents referred to here as well as other publications known to those skilled in the art the micro needles of the array are generally positioned in a uniform pattern so that each micro needle is placed at a distance which is substantially the same as its distance away from the next closest needle. The above-referenced patents as well as patents and publications disclosed and cited in each of these patents are incorporated herein by reference in their entirety in order to disclose and describe arrays of micro needles as well as methods of using and manufacturing such arrays.

In an embodiment of the invention, the distance between one micro needle and the next closest micro needle, e.g. micro needles 102 and 104 is in the range of 10 μm to 200 μm. The length of a micro needle, for example micro needle 102, is selected in a manner that ensures that the micro needle gets past the stratum cornea, i.e., the upper-most layer of the skin while not penetrating—deep enough to contact the nerve receptors. In one embodiment of the invention, the length of micro needle 102 is in the range of 1 micron to 1000 microns. In another embodiment of the invention, the length of micro needle 102 is in the range of 50 to 400 microns.

Each micro needle, for example, micro needle 102 has a tip, which facilitates insertion of micro needle 102 into the body of the subject. The diameter of the tip of micro needle 102 is selected on the basis of various factors, including its manufacture, durability, and ability to get coated with the neurotoxin. The diameter of the tip of micro needle 102 should be such that an adequate surface area is available for coating micro needle 102 with a sufficient amount of the neurotoxin to cause a therapeutic effect.

In an embodiment of the invention, the diameter of the tip of micro needle 102 is in the range of 1 μm to 5 μm. Micro needle 102 is pre-coated with the neurotoxin. The neurotoxin is coated on the tip of micro needle 102. For example, micro needle 102 is coated with botulinum toxin. In an embodiment of the invention, micro needle 102 is coated with the neurotoxin in dry form. The area of micro needle 102 pre-coated with the neurotoxin varies from 1 to 100 percent of the surface area of micro needle 102. Each micro needle of array of micro needles 100 is so coated that the cumulative amount of the coated neurotoxin is equal to a pre-defined amount of the neurotoxin. The pre-defined amount of the neurotoxin is calculated on the basis of the amount of the neurotoxin to be delivered into the skin of the subject and the assumed losses of the neurotoxin during the manufacture, storage and handling of array of micro needles 100, during hydration and release from the array of micro needles 100 after its application to the skin's surface.

There are various methods for coating micro needles 102, 104, 106, and 108. In an embodiment of the invention, array of micro needles 100 is dipped into a liquid solution of the neurotoxin. The neurotoxin is then allowed to dry on the tip of micro needles 102, 104, 106, and 108. In another embodiment of the invention, the neurotoxin is applied onto micro needles 102, 104, 106 and 108 and lyophilized. Micro needle 102 delivers an amount of the neurotoxin ranging from 1 to 25 units. An array of micro needles 100 delivers an amount of the neurotoxin ranging from 10 to 250 units. The neurotoxin may include active pharmaceutical ingredients mixed with other excipients, i.e. inactive carriers. In an embodiment of the invention, the excipients are selected from standard excipients such as sugars, polyols, salts, buffers, and surfactants. However, the invention should not be construed to be limited to the use of a particular formulations of carrier with the neurotoxin. Various possible combinations of one or more of active pharmaceutical ingredients, excipients and other necessary ingredients may be used without deviating from the scope of the invention.

### Delivery Method

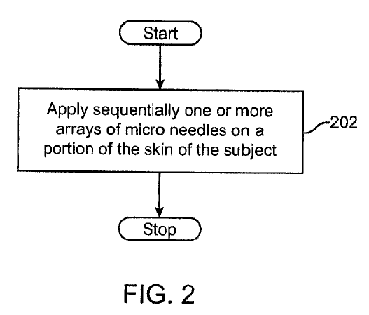

FIG. 2 is a flowchart of a method for delivering a neurotoxin into the subject, in accordance with an embodiment of the invention. A neurotoxin is delivered to treat disorders such as axillary hyperhidrosis, palmar hyperhidrosis, warts, corns, calluses, neuromas, ulcers, hammertoes, bunions, and post herpetic neuralgia.

At step 202, one or more arrays of micro needles 100 are applied sequentially on a portion of the skin of the subject affected by the disorder. Micro needles 102, 104, 106 and 108 are pre-coated with the neurotoxin. The array of micro needles 100 has been explained in conjunction with FIG. 1. In one embodiment of the invention, the number of arrays of micro needles, applied on the portion of the skin of the subject, is determined on the basis of the amount of the neurotoxin to be delivered to the target area of the subject and the amount each array of micro needles can deliver.

In another embodiment of the invention, the number of arrays of micro needles, applied on the portion of the skin of the subject, is determined based on the affected area of the skin of the subject and the area to which array of micro needles is applied. For example, if the affected area of the skin of the subject is 100 mm² and one micro needle is capable of treating 25 mm², then four micro needles are required to treat the affected area of the skin of the subject. In various embodiments of the invention, the number of arrays of micro needles applied to the portion of the skin varies from one to ten.

In an embodiment of the invention, the neurotoxin is mixed with an enhancing agent to enhance the permeability of the portion of the skin for delivery of the neurotoxin through the portion of the skin. The range of surface area of the portion of the skin to which the arrays of micro needles are applied is in the range of 1 mm² to 200 mm². The array of micro needles 100 is applied for a pre-determined time. The pre-determined time depends on the rate of delivery of the neurotoxin to the target area, e.g. through the portion of the skin. The pre-determined time ranges from 1 second to 30 minutes. In an embodiment of the invention, the range of the pre-determined time is between 5 seconds to 10 minutes.

The application of one or more arrays of micro needles results in the delivery of the neurotoxin into the subject. Micro needles 102, 104, 106 and 108 pierce a portion of the skin and create micro conduits for the transport of the neurotoxin across the portion of the skin. As a result, the neurotoxin coated on micro needle 102 is delivered into the body of the subject. Micro needles 102, 104, 106 and 108 penetrate only the top layers of the skin and allow the neurotoxin to pass through with ease. However, micro needles 102, 104, 106 and 108 do not, in general, penetrate deep enough to contact and stimulate nerves. As a result, the insertion of micro needles 102, 104, 106 and 108 is intended to be painless and bloodless. For example, the micro needles on the surface of Dermaroller™ C-8 create miniscule holes of 0.07 mm diameter and 0.13 mm in depth, while a cylinder present on the micro needle mechanically pushes the applied substance inside the holes and finally under the portion of the skin.

The use of one or more arrays of micro needles 100 results in an improved process of delivering the neurotoxin. The neurotoxin penetrates the area (e.g. into the portion of the skin of the subject) and provides the required amount of the neurotoxin uniformly to the target area (e.g. under the surface of the skin). Micro needles can be painlessly applied on the portion of the skin of the subjects and may not require any medical expertise. Moreover, the use of one or more arrays of micro needles reduces the time of treatment for delivering the neurotoxin. This is because a higher amount of the neurotoxin is delivered in a shorter period of time. The use of micro needles enhances delivery of the neurotoxin across the portion of the skin. Further, the process of delivering the neurotoxin is less distressing for the subject.

While the preferred embodiments of the invention have been illustrated and described, it will be clear that the invention is not so limited. Numerous modifications, changes, variations, substitutions and equivalents will occur to those skilled in the art without departing from the spirit and scope of the present invention, as defined by the appended claims.

What is claimed is:

1. A system for delivering a neurotoxin into a subject, the system comprising:
an array of micro needles, each micro needle being pre-coated with a neurotoxin.

2. The system of claim 1, wherein the neurotoxin is botulinum toxin.

3. The system of claim 1, wherein length of each micro needle is selected such that the micro needle penetrates the stratum cornea and does not penetrate deep enough to contact nerve receptors and the system comprises a plurality of arrays for sequential application.

4. The system of claim 1, wherein length of each micro needle is between 1 micron and 1000 microns.

5. The system of claim 1, wherein length of each micro needle is between 50 microns and 400 microns.

6. The system of claim 1, wherein each micro needle is coated with neurotoxin over a surface area representing 1% to 100% of the surface area of each micro needle.

7. The system of claim 19, wherein micro needles are positioned on the system over an area of between 1 mm² and 200 cm².

8. The system of claim 1, designed for treatment of a disorder selected from a group consisting of axillary hyperhidrosis, palmar hyperhidrosis, warts, corns, calluses, neuromas, ulcers, hammertoes, bunions and post herpetic neuralgia.

9. The system of claim 1, wherein the amount of the neurotoxin delivered is between 1 unit and 25 units.

10. The system of claim 1, further comprising:
a base interconnecting the micro needles.

11. The system of claim 10, wherein the based is substantially planar.

12. The system of claim 11, wherein the base is flexible.

13. The system of claim 11, wherein the base has thereon 10 to 1,000 needles over an area of from about 1 mm² to 4 cm².

14. The system of claim 13, wherein the micro needles are 50 microns to 400 microns in length.

15. A method of administering a neurotoxin to a target area of a patient, comprising the steps of:
(a) applying an array of micro needles to a target area of a patient wherein the micro needles are coated with a therapeutically effective amount of a formulation comprised of a neurotoxin; and
(b) allowing the micro needles to remain in the target area for a period of time such that neurotoxin is delivered to the target area.

16. The method of claim 15, further comprising:
(c) repeating steps (a) and (b) a plurality of times using a new array of micro needles in each step (a).

17. The method of claim 15, wherein micro needles of the array are sequentially applied to the target area wherein the target area is an area below an outer layer of human skin and wherein the neurotoxin is botulinum toxin.

18. The method of claim 17, wherein an array of micro needles is applied for a time which is determined based on the rate of delivery of the neurotoxin through the portion of the skin.

19. The method of claim 15, wherein an array of micro needles is applied for a period between 1 second and 30 minutes.

20. The method of claim 15, wherein an array of micro needles is applied for a period between 5 seconds and 10 minutes and wherein the length of a micro needle is selected such that the micro needle gets past the stratum cornea and does not penetrate deep enough to contact the nerve receptors.

21. The method of claim 15, wherein length of a micro needle is between 1 micron and 1000 microns.

22. The method of claim 15, wherein length of a micro needle is between 50 microns and 400 microns and wherein the micro needles are coated over an area of 1% to 100% of the surface area of the micro needle.

23. The method of claim 15, wherein the surface area of the portion of the skin is between 1 mm² and 200 cm².

24. The method of claim 15, wherein the neurotoxin is delivered to treat one or more disorders selected from a group including axillary hyperhidrosis, palmar hyperhidrosis, warts, corns, calluses, neuromas, ulcers, hammertoes, bunions and post herpetic neuralgia.

25. The method of claim 16, wherein the number of arrays of micro needles that are applied sequentially is determined based on the amount of the neurotoxin to be delivered and the amount that each array of micro needles can deliver.

26. The method of claim 16, wherein the number of arrays of micro needles that are applied sequentially is determined based on the area of the affected portion of the skin of the subject and the area to which an array of micro needles is applied.

27. The method of claim 16, wherein in step (c) 1 to 10 new arrays of micro needles are sequentially applied to the target area which is a portion of skin of the patient.

28. The method of claim 27, wherein the amount of the neurotoxin delivered to the subject by all the arrays of micro needles applied sequentially is between 10 and 250 units.
